(19) United States
(12) Patent Application Publication
(10) Pub. No.: US 2019/0135761 A1
(43) Pub. Date: May 9, 2019

(54) COMPOSITION COMPRISING 3-(HALOALKYL OR FORMYL)-1H-PYRAZOLE-4-CARBOXYLIC ACIDS OR ESTERS, ITS MANUFACTURE AND ITS USE FOR THE PREPARATION OF CARBOXAMIDES

(71) Applicant: SOLVAY SA, Brussels (BE)

(72) Inventor: Janis JAUNZEMS, Hannover (DE)

(21) Appl. No.: 16/099,804

(22) PCT Filed: May 9, 2017
(86) PCT No.: PCT/EP2017/061020

§ 371 Date: Nov. 8, 2018

(30) Foreign Application Priority Data
May 10, 2016 (EP) 16169005.2

(51) Int. Cl. C07D 231/14

(52) U.S. Cl. CPC C07D 231/14

(57) ABSTRACT

The present invention concerns compositions comprising (i) a 3-(haloalkyl or formyl)-1H-pyrazole wherein R¹ is a halogenated C₁₋₄ alkyl group, or the group —C(O)H, R² is selected from the group consisting of H, C₁-C₁₂ alkyl, C₂-C₆ alkenyl, cycloalkyl, aryl, heteroaryl, aralkyl, each of which is optionally substituted, R³ is selected from the group consisting of H, C₁-C₁₂ alkyl, C₂-C₆ alkenyl, aryl or C₃-C₈ cycloalkyl, each of which is optionally substituted, and R⁴ is selected from the group consisting of H, X′, COOR′, OR′, SR′, C(O)NR′₂, and (ii) at least one of the pyrazole derivatives of the formulae (II)-(X), (XVIIIa), (XVIIIb), (XIX) and (XX), wherein the chemical composition comprises equal to or more than 95 w % of compound of formula (I), its manufacture and use in processes for the manufacture of agrochemical or pharmaceutical pyrazole-4-carboxamide compounds.

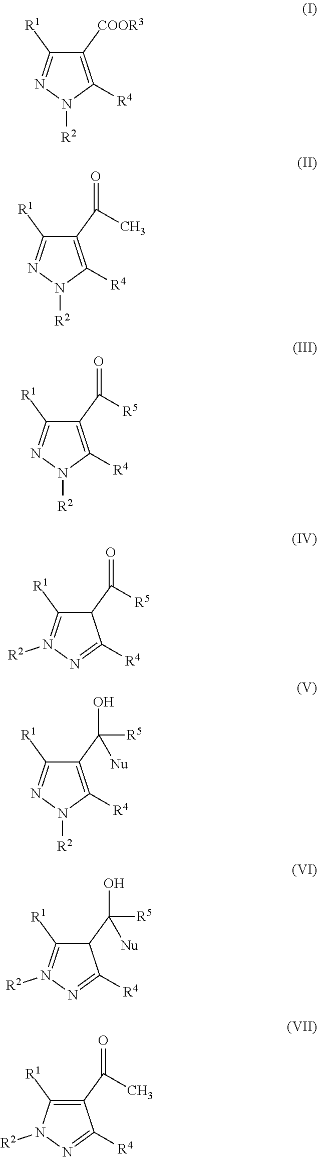

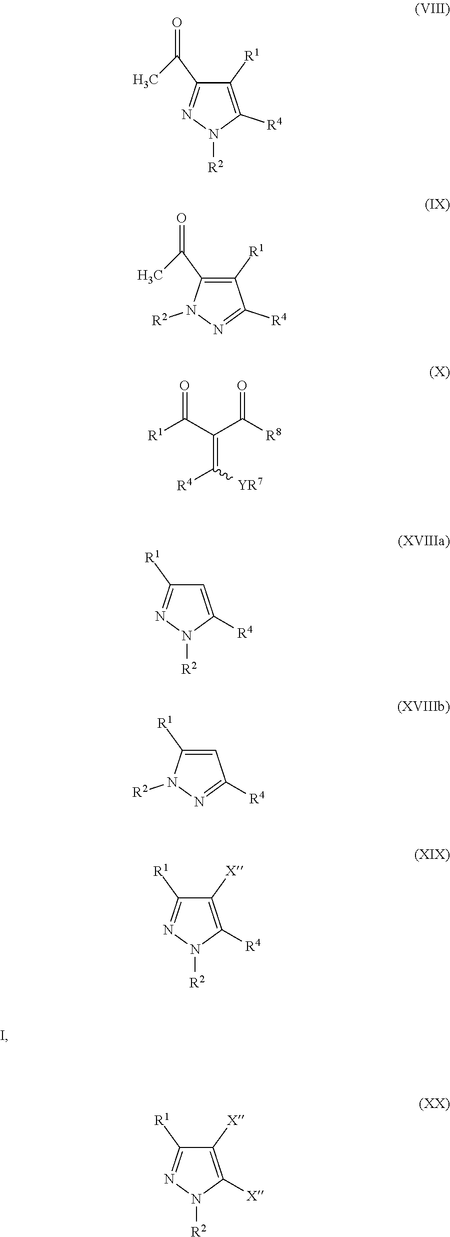

This application claims priority to European application No. 16169005.2, the whole content of this application being incorporated herein by reference for all purposes.

The present invention concerns compositions comprising a haloalkyl pyrazole, its manufacture and use in processes for the manufacture of agrochemical or pharmaceutical compounds.

3-halomethylpyrazole-4-yl carboxylic acids and esters are valuable intermediates in the synthesis of agrochemical and pharmaceutical active ingredients. Agrochemical active ingredients which contain such pyrazole building blocks are, for example, 2′-[1,1′-bicycloprop-2-yl]-3-(difluoromethyl)-1-methylpyrazole-4-carboxanilide (Sedaxane), as described, for example, in WO2006015866, 3-(difluoromethyl)-1-methyl-N[2-(3′,4′,5′-trifluorophenyl)phenyl]pyrazole-4-carboxamide (Fluxapyroxad), as described, for example, in WO2006087343, N-(3′,4′-Dichloro-5-fluorobiphenyl-2-yl)-3-(difluoromethyl)-1-methylpyrazole-4-carboxamide (Bixafen), as described, for example, in WO2003070705, 3-(Difluoromethyl)-1-methyl-N-[1,2,3,4-tetrahydro-9-(1-methylethyl)-1,4-methanonaphthalen-5-yl]-1H-pyrazole-4-carboxamide (Isopyrazam), as described, for example, in WO2004035589, (RS)-N-[9-(Dichloromethylen)-1,2,3,4-tetrahydro-1,4-methanonaphthalin-5-yl]-3-(difluoromethyl)-1-methyl-1H-pyrazole-4-carboxamide (Benzovindiflupyr), as described, for example, in WO07048556. Generally, 3-halomethylpyrazole-4-yl carboxylic acids, often obtained by hydrolysis of their esters, are converted into the carboxamides, for example after conversion into the 3-halomethylpyrazole-4-yl carboxylic acid halide. Other conversions, wherein the carboxamide is generated directly from the ester or acid, have also been described, such as in WO2012055864 and WO 2007/031323. All foregoing cited patent applications are hereby incorporated for all purposes.

The pyrazoles building blocks which are used as intermediates for the manufacture of said agrochemical and pharmaceutical active ingredients are often manufactured by processes which render the compositions comprising the intermediary pyrazoles impure with respect to side-products and regioisomers. As these impurities often interfere with downstream processes and are difficult to remove, manufacture of the intermediary pyrazole building blocks has thus been subject to many process improvements to circumvent complex compositions of such pyrazoles, see, for example, US2011046371. It has now been found that compositions according to the present invention comprising haloalkyl pyrazole building blocks are particularly well suited in the manufacture of downstream products, in particular carboxamide based agrochemical and pharmaceutical active ingredients.

The invention thus concerns chemical compositions comprising a compound of formula (I)

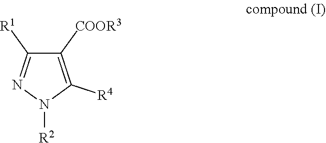

wherein

R¹ is a halogenated C₁₋₄ alkyl group, or R¹ is the group —C(O)H

R² is selected from the group consisting of H, C₁-C₁₂-alkyl, C₂-C₆ alkenyl, cycloalkyl, aryl, heteroaryl, aralkyl, each of which is optionally substituted,

R³ is selected from the group consisting of H, C₁-C₁₂-alkyl, C₂-C₆ alkenyl, aryl or C₃-C₈-cycloalkyl group, each of which is optionally substituted and

R⁴ is selected from the group consisting of H, X′, COOR′, OR′, SR′, C(O)NR′₂, wherein R′ are selected independently in C(O)NR′₂ where R′ is hydrogen or a C₁-C₁₂-alkyl group, CN, C₁-C₁₂-alkyl, C₂-C₆ alkenyl, aryl, cycloalkyl, aralkyl, heteroaryl, each of which is optionally substituted;

and at least one of the compounds of formula (II) to (X), which will be disclosed in more detail below,

wherein the chemical composition comprises equal to or more than 95 w % (content c1) of compound of formula (I).

The invention also relates to a process for the manufacture of a chemical composition according to the present invention, which comprises at least one of the steps of subjecting a chemical composition comprising a compound of formula (I) which has a content of compound of formula (I) of c2>c1, to at least one step selected from the group of steps consisting of:

i. crystallization

ii. washing

iii. chromatography

iv. salt formation, recovery of intermediary salt, and hydrolysis of salt

v. distillation,
wherein c1 and c2 will be disclosed in more detail below.

A further subject of the present invention are compounds of formulae (III), wherein in compound (III) R⁵ is a monohalo- or dihalomethyl group or wherein R⁵ is the group N(R²)₂, wherein the two R² groups can be independently selected, to (XI), which will be disclosed below.

The invention further concerns a process for the manufacture of an agrochemical or pharmaceutical compound, which comprises the step of reaction of the chemical composition according to the present invention with at least one amine of formula (VI) NR¹²HQ, wherein R¹² is selected from the group consisting of H, C₁-C₁₂-alkyl, C₂-C₆ alkenyl or C₃-C₈-cycloalkyl group, wherein H and C₁-C₄-alkyl are preferred, and wherein Q is an optionally substituted aryl or heteroaryl group.

The chemical composition according to the present invention comprises a compound of formula (I)

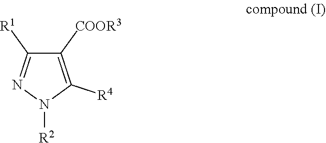

wherein

R¹ is a halogenated C₁₋₄ alkyl group, or R1 is the group —C(O)H

R² is selected from the group consisting of H, C₁-C₁₂-alkyl, C₂-C₆ alkenyl, cycloalkyl, aryl, heteroaryl, aralkyl, each of which is optionally substituted,

R³ is selected from the group consisting of H, C₁-C₁₂-alkyl, C₂-C₆ alkenyl, aryl or C₃-C₈-cycloalkyl group, each of which is optionally substituted and

R⁴ is selected from the group consisting of H, X′, COOR′, OR′, SR′, C(O)NR′₂, wherein R′ are selected independently in C(O)NR′₂ where R′ is hydrogen or a C₁-C₁₂-alkyl group, CN, C₁-C₁₂-alkyl, C₂-C₆ alkenyl, aryl, cycloalkyl, aralkyl, heteroaryl, each of which is optionally substituted;

and at least one of the compounds of the group consisting of:

### Formula (II)

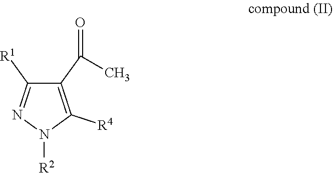

### Formula (III)

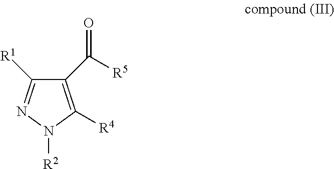

wherein R⁵ is a halogenated methyl group or wherein R⁵ is the group N(R²)₂, wherein the two R² groups can be independently selected from the group defined above for R²,

### Formula (IV)

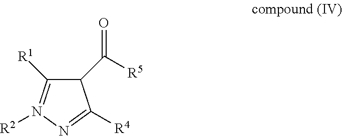

wherein R¹, R², R⁴ and R⁵ have the same meaning as above,

### Formula (V)

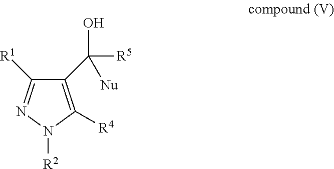

wherein R¹, R², R⁴ and R⁵ have the same meaning as above, and wherein Nu is a nucleophilic group, particularly NR⁶R^{6′} or OR⁶, wherein R⁶ is selected from the group consisting of H, C₁-C₁₂-alkyl, C₂-C₆ alkenyl, cycloalkyl, aryl, heteroaryl, aralkyl, each of which is optionally substituted, or wherein R⁶ and R^{6′} together with the nitrogen atom to which they are attached form a 3-7 membered heterocyclic ring which can be saturated or unsaturated,

### Formula (VI)

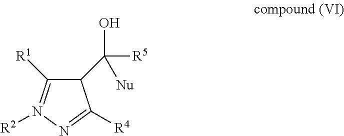

wherein R¹, R², R⁴, R⁵ and R⁶ have the same meaning as above,

### Formula (VII)

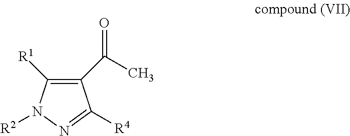

wherein R¹, R² and R⁴ have the same meaning as above,

### Formula (VIII)

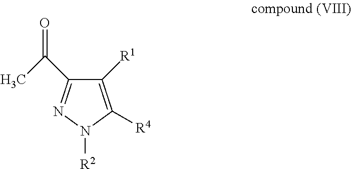

wherein R¹, R² and R⁴ have the same meaning as above,

### Formula (IX)

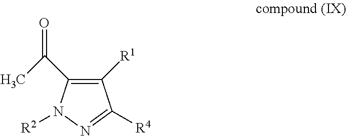

wherein R¹, R² and R⁴ have the same meaning as above,

### Formula (X)

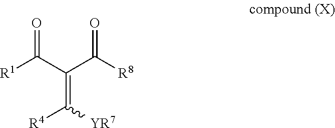

wherein R¹ and R⁴ are described as above, R⁸ is R⁵ or CH₃, Y is selected from the group consisting of S, O and NR⁹, wherein O and NR⁹ are preferred,

wherein R⁷ and R⁹ independently are selected from the group consisting of C₁-C₁₂-alkyl, C₂-C₆ alkenyl or C₃-C₁₀-cycloalkyl group, each of which is optionally substituted

or, when Y=NR⁹, R⁷ together with R⁹ and the nitrogen atom to which the two radicals are attached are an optionally substituted 5- to 10-membered heterocyclic radical which, in addition to the nitrogen atom, may contain a further 1, 2 or 3 heteroatoms selected from the group consisting of O, N and S as ring members

### Formula (XVIIIa)

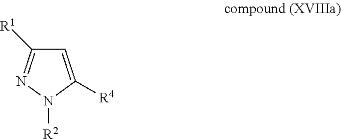

wherein R¹, R² and R⁴ are defined as above,

### Formula (XVIIIb)

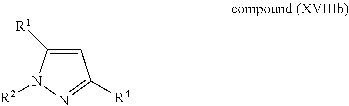

wherein R¹, R² and R⁴ are defined as above,

### Formula (XIX)

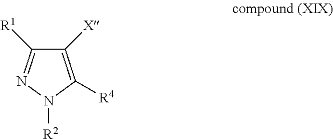

wherein R¹ and R⁴ are defined as above, and X″ is selected from the group consisting of Br, Cl and I,

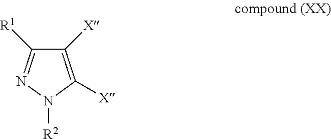

wherein R¹ is defined as above, and X″ is independently selected from the group consisting of Br, Cl and I,

and optionally a compound of formula (XI)

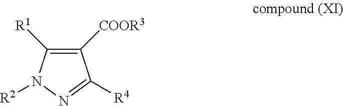

wherein R¹, R² and R⁴ have the same meaning as above, wherein the content of (XI), if present, is equal to or less than 0.9%,

wherein the chemical composition comprises equal to or more than 95 w % (content c1) of compound of formula (I).

According to the present invention, R¹ is a halogenated C₁₋₄ alkyl group, wherein in the term “C₁₋₄ alkyl group” means a group selected from the group consisting of methyl, ethyl, i-propyl, n-propyl, n-butyl, tert-butyl and sec.-butyl, wherein methyl is preferred. The term “halogenated C₁₋₄ alkyl group” intends to denote that the C₁₋₄ alkyl group is substituted by one or more halogen atoms, preferably selected from F, Cl and Br, preferably F and Cl. The halogenated C₁₋₄ alkyl group can also be substituted by other substituents, such as, for example, CN or OH. Preferably, R¹ is selected from the group CF₂Cl, CF₂H, CFCl₂, CFClH, CF₂Br, CCl₃, CF₃, CBr₃, and CI₃. More preferably, R¹ is selected from the group consisting of CF₂Cl, CF₂H, CFCl₂, CFClH and CF₂Br. Even more preferably, R¹ is selected from the group consisting of CF₂Cl, CF₂H, CFCl₂ and CFClH. Most preferably, R¹ is selected from the group consisting of CF₂Cl and CF₂H. In a most particularly preferred aspect, R¹ is CF₂H. In another aspect, R¹ is the group —C(O)H. The group —C(O)H can be obtained, for example, from the group R¹ which is a halogenated C¬₁₋₄ alkyl group which is hydrolysed, for example in a previous reaction step to obtain the composition of the present invention.

According to the present invention, R² is selected from the group consisting of H, C₁-C₁₂-alkyl, C₂-C₆ alkenyl, C₃-C₁₀-cycloalkyl, aryl, heteroaryl, aralkyl, each of which is optionally substituted by one or more groups selected from the group consisting of —R′, —X′, —OR′, —SR′, —NR′₂, —SiR′₃, —COOR″, —CN and —CONR′₂, where R′ is hydrogen or a C₁-C₁₂-alkyl group which are the same or different in —CONR′₂, and X′ is F, Cl, Br, or I. Most preferably, R² is methyl.

For the purpose of the present invention, the definition C₁-C₁₂-alkyl comprises the largest range defined herein for an alkyl group. Specifically, this definition comprises, for example, the meanings methyl, ethyl, n-propyl, isopropyl, n-, iso-, sec- and t-butyl, n-pentyl, n-hexyl, 1,3-dimethylbutyl, 3,3-dimethylbutyl, n-heptyl, n-nonyl, n-decyl, n-undecyl and n-dodecyl. Often, methyl, ethyl, n-propyl, isopropyl, n-, iso-, sec- and t-butyl are most preferred residues selected from the group C₁-C₁₂-alkyl.

The term “C₃-C₁₀-cycloalkyl”, as used in this invention, denotes mono-, bi- or tricyclic hydrocarbon groups comprising 3 to 10 carbon atoms, especially 3 to 6 carbon atoms. Examples of monocyclic groups include cyclopropyl, cyclobutyl, cyclopentyl, cyclohexyl, cycloheptyl or cyclooctyl. Examples of bicyclic groups include bicyclo[2.2.1]heptyl, bicyclo[3.1.1]heptyl, bicyclo[2.2.2]octyl and bicyclo[3.2.1]octyl. Examples of tricyclic groups are adamantyl and homoadamantyl.

In the context of the present invention, aryl groups are, unless defined otherwise, aromatic hydrocarbon groups which may contain one, two or more heteroatoms selected from the group consisting of O, N, P and S and which may optionally be substituted by further groups selected from the group consisting of R′, —X′, —OR′, —SR′, —NR′₂, —SiR′₃, —COOR′, —(C—O)R′, —CN and —CONR′₂, where R′ and X′ are defined as above.

In one aspect, the term “aryl” is a C₅-C₁₈-aryl. The term “C₅-C₁₈-aryl” denotes the largest range defined herein for an aryl groups having 5 to 18 skeleton atoms, where the carbon atoms may be replaced by heteroatoms, thus forming a heteroaryl. Specifically, this definition comprises, for example, the meanings cyclopentadienyl, phenyl, cycloheptatrienyl, cyclooctatetraenyl, naphthyl and anthracenyl; 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 1-pyrrolyl, 1-pyrazolyl, 1,2,4-triazol-1-yl, 1-imidazolyl, 1,2,3-triazol-1-yl, 1,3,4-triazol-1-yl; 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

In the context of the present invention, arylalkyl groups (aralkyl groups) are, unless defined otherwise, alkyl groups which are substituted by aryl groups, which may have a C₁₋₈-alkylene chain and which may be substituted in the aryl skeleton or the alkylene chain by one or more heteroatoms selected from the group consisting of O, N, P and S and optionally by further groups selected from the group consisting of R′, —X′, —OR′, —SR′, —NR′₂, —SiR′₃, —COOR′, —(C—O)R′, —CN and —CONR′₂, where R′, which may further contain one or more heteroatoms selected from the group consisting of N, O, P and S, and X′ are defined as above.

The definition C₇-C₁₉-aralkyl group comprises the largest range defined herein for an arylalkyl group having a total of 7 to 19 atoms in the skeleton and the alkylene chain. Specifically, this definition comprises, for example, the meanings benzyl and phenylethyl.

The term “C₂-C₆-alkenyl group” denotes a group comprising a carbon chain and at least one double bond. Alkenyl group are, for example, ethenyl, propenyl, butenyl, pentenyl or hexenyl.

R³ is selected from the group consisting of H, C₁-C₁₂-alkyl, C₂-C₆ alkenyl, aryl or C₃-C₈-cycloalkyl group, each of which is optionally substituted by one or more groups selected from the group consisting of R′, —X′, —OR′, —SR′, —NR′₂, —SiR′₃, —COOR′, —(C—O)R′, —CN and —CONR′₂, where R′, which may further contain one or more heteroatoms selected from the group consisting of N, O, P and S, and X′ are defined as above. Preferably, R³ is H, Me or Et. In one preferred aspect, R³ is H.

R⁴ is selected from the group consisting of H, X′, COOR′, OR′, SR′, C(O)NR′₂, wherein R′ are selected independently in C(O)NR′₂ where R′ is hydrogen or a C₁-C₁₂-alkyl group, CN, C₁-C₁₂-alkyl, C₂-C₆ alkenyl, aryl, cycloalkyl, aralkyl, heteroaryl, each of which is optionally substituted by one or more groups selected from the group consisting of —R′, —X′, —OR′, —SR′, —NR′₂, —SiR′₃, —COOR′, —(C—O)R′, —CN and —CONR′₂, in which R′ are selected independently, wherein R′ is hydrogen or a C₁-C₁₂-alkyl group and X′ is F, Cl, Br, or I; when R⁴ is a C₁-C₁₂-alkyl group, methyl, ethyl, n-propyl, isopropyl, n-, iso-, sec- and t-butyl are preferred, and methyl and ethyl are most preferred; when R⁴ is X′, Br and F are most preferred. In a most preferred aspect, R⁴ is H.

According to the present invention, R⁵ is a halogenated methyl group, for example a methyl group substituted with one, two or three halogen atoms, which independently are selected from the group consisting of F, Cl, Br and I. For example, R⁵ is selected from the group consisting of CF₃, CCl₃, CBr₃ and CI₃. Preferably, R⁵ is CCl₃ or CBr₃. R⁵ can further be selected from the group consisting of trihalomethyl, wherein at least one halogen atom is different than at least one other halogen atom with which methyl is substituted, for example CBr₂F or CClF₂. R⁵ can also be selected from the group consisting of dihalomethyl groups, for example CF₂H, CCl₂H, CBr₂H and Cl₂H. Preferably, the dihalomethyl group is CCl₂H or CBr₂H. The dihalomethylgroup can also contain two halogen atoms of a different species, for example CBrClH. R⁵ can further be selected from the group consisting of monohalomethyl groups, for example CH₂F, CH₂Br, CH₂Cl and CH₂I. Preferably, the monohalomethyl group is CH₂Br or CH₂Cl. In another aspect, R⁵ can also be a group N(R²)₂, wherein the two R² groups can be independently selected from the group defined above for R². N(R²)₂ can be, for example, NH₂ or at least one R² can be a C₁-C₁₂-alkyl group. R⁵ being N(R²)₂ can be achieved, for example, by a reagent, solvent or catalyst being present in any previous reaction step to obtain the composition according to the present invention, wherein reagent, solvent or catalyst being present bears a group —N(R²)₂.

According to the present invention, Nu is a nucleophilic group. In particular, Nu is NR⁶R^{6′} or OR⁶, wherein R⁶ is selected from the group consisting of H, C₁-C₁₂-alkyl, C₂-C₆ alkenyl, cycloalkyl, aryl, heteroaryl, aralkyl, each of which is optionally substituted, or wherein R⁶ and R^{6′} together with the nitrogen atom to which they are attached form a 3-7 membered heterocyclic ring which can be saturated or unsaturated. When Nu is OR⁶, R⁶=H is preferred.

According to the present invention, R⁸ is R⁵ or CH₃, Y is selected from the group consisting of S, O and NR⁹, wherein O and NR⁹ are preferred,

wherein R⁷ and R⁹ independently are selected from the group consisting of C₁-C₁₂-alkyl, C₂-C₆ alkenyl or C₃-C₁₀-cycloalkyl group, each of which is optionally substituted by one or more groups selected from the group consisting of —R′, —X′, —OR′, —SR′, —NR′₂, —SiR′₃, —COOR′, —(C—O)R′, —CN and —CONR′₂, in which R′ are selected independently, wherein R′ is hydrogen or a C₁-C₁₂-alkyl group and X′ is F, Cl, Br, or I

or, when Y=NR⁹, R⁷ together with R⁹ and the nitrogen atom to which the two radicals are attached are an optionally substituted 5- to 10-membered heterocyclic radical which, in addition to the nitrogen atom, may contain a further 1, 2 or 3 heteroatoms selected from the group consisting of O, N and S as ring members. For R⁷ and R⁹, ethyl is preferred.

The present invention further concerns the compounds of formulae (III) to (XI) as disclosed above, with the proviso that in (III) and (X), R⁵ is a dihalo- or monohalomethyl group or wherein R⁵ is the group N(R²)₂, wherein the two R² groups can be independently selected from the group defined above for R². The monohalomethyl group is selected from the group consisting of CH₂Cl, CH₂Br, CH₂F and CH₂I, wherein CH₂Cl and CH₂Br are preferred. The dihalomethyl group is selected from the group consisting of CCl₂H, CBr₂H, CF₂H and CI₂H, wherein CCl₂H and CBr₂H are preferred.

As disclosed above, the chemical composition comprises equal to or more than 95 w % (content c1) of compound of formula (I). In a preferred aspect, the chemical composition comprises equal to or more than 98 w % (content c1) of compound of formula (I). In another preferred aspect, the chemical composition comprises equal to or more than 99 w % (content c1) of compound of formula (I).

The content c1 is determined by methods of analysis for organic compounds well known to the person skilled in the art, in particular NMR (nuclear magnetic resonance; ¹H, ¹³C or other nuclei; one- or two-dimensional), mass spectrometry, which can be coupled with HPLC (high performance liquid chromatography), LQ (liquid chromatography) or GC (gas chromatography), elemental analysis, HPLC, GC or IR (infrared spectroscopy). Melting points, decomposition points or boiling points can also be suitable analytical methods. Cl is expressed in weight percent (w %). The term ppmw denotes, for example that 0.1 w % is 1000 ppmw, and 1 w % is 10.000 ppmw.

According to the present invention, the cumulative amount of compounds (II) to (XI) comprised in the chemical composition is equal to or less than 5 w %, preferably equal to or less than 2 w % and more preferably equal to or less than 1 w %. The cumulative amount of compounds (II) to (XI) can be determined by the same analytical technologies as c1. In a particular aspect, the cumulative amount of compounds (II) to (XI) comprised in the chemical composition is equal to or less than 10.000 ppmw, preferably equal to or less than 5.000 ppmw, and more preferably equal to or less than 1.000 ppmw.

In one aspect of the present invention, the individual amount of compounds (II) to (XI) comprised in the chemical composition is equal to or less than 1.000 ppmw, preferably equal to or less than 700 ppmw, more preferably equal to or less than 400 ppmw and most preferably equal to or less than 200 ppmw. The individual amount of compounds (II) to (XI) can be determined by the same technologies as c1. For lower amounts of compounds (II) to (XI), MS, optionally coupled with HPLC or GC, is used preferentially, in particular when contents of equal to or lower than 100 ppmw are concerned.

If one or more compounds of formula (XI)

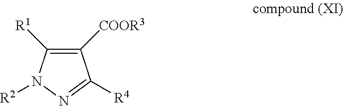

wherein R¹, R² and R⁴ have the same meaning as above, are present, the content of (XI), is equal to or less than 0.9%. The content of (XI) can also be equal to or less than 0.8%, or equal to or less than 0.7%, or equal to or less than 0.6%, or equal to or less than 0.5%, or equal to or less than 0.4%, or equal to or less than 0.4%, or equal to or less than 0.3%, or equal to or less than 0.2%, or equal to or less than 0.1%. The content of (XI) is measured suitably by ¹H-NMR. A content of equal to or less than 0.5% is preferred, and a content of equal to or less than 0.3% is more preferred.

Table 1 discloses preferred compounds of formula (I).

TABLE 1

| Compound # | R¹ | R² | R³ | R⁴ |
|---|---|---|---|---|
| I.1 | CF₃ | Me | H | H |
| I.2 | CF₂H | Me | H | H |
| I.3 | CF₂Cl | Me | H | H |
| I.4 | CF₂Br | Me | H | H |

Table 2 discloses preferred compounds of formula (II), (VII), (VIII) and (IX).

TABLE 2

| Compound # | R¹ | R² | R⁴ |
|---|---|---|---|
| II/VII/VII/IX.1 | CF₃ | Me | H |
| II/VII/VII/IX.2 | CF₂H | Me | H |
| II/VII/VII/IX.3 | CF₂Cl | Me | H |
| II/VII/VII/IX.4 | CF₂Br | Me | H |

Table 3 discloses preferred compounds of formula (III) and (IV).

TABLE 3

| Compound # | R¹ | R² | R⁴ | R⁵ |
|---|---|---|---|---|
| III/IV.1 | CF₃ | Me | H | CCl₃ |
| III/IV.2 | CF₃ | Me | H | CBr₃ |
| III/IV.3 | CF₃ | Me | H | CHCl₂ |
| III/IV.4 | CF₃ | Me | H | CHBr₂ |
| III/IV.5 | CF₃ | Me | H | CH₂Br |
| III/IV.6 | CF₃ | Me | H | CH₂Cl |
| III/IV.7 | CF₂H | Me | H | CCl₃ |
| III/IV.8 | CF₂H | Me | H | CBr₃ |
| III/IV.9 | CF₂H | Me | H | CHCl₂ |
| III/IV.10 | CF₂H | Me | H | CHBr₂ |
| III/IV.11 | CF₂H | Me | H | CH₂Br |
| III/IV.12 | CF₂H | Me | H | CH₂Cl |
| III/IV.13 | CF₂Cl | Me | H | CCl₃ |
| III/IV.14 | CF₂Cl | Me | H | CBr₃ |
| III/IV.15 | CF₂Cl | Me | H | CHCl₂ |
| III/IV.16 | CF₂Cl | Me | H | CHBr₂ |
| III/IV.17 | CF₂Cl | Me | H | CH₂Br |
| III/IV.18 | CF₂Cl | Me | H | CH₂Cl |
| III/IV.19 | CF₂Br | Me | H | CCl₃ |
| III/IV.20 | CF₂Br | Me | H | CBr₃ |
| III/IV.21 | CF₂Br | Me | H | CHCl₂ |
| III/IV.22 | CF₂Br | Me | H | CHBr₂ |
| III/IV.23 | CF₂Br | Me | H | CH₂Br |
| III/IV.24 | CF₂Br | Me | H | CH₂Cl |

Table 4 discloses preferred compounds of formula (V) and (VI).

TABLE 4

| Compound # | R¹ | R⁴ | R⁸ | YR⁷ |
|---|---|---|---|---|
| V/VI.1 | CF₃ | H | CH₃ | Y = O, R⁷ = Et |
| V/VI.2 | CF₃ | H | CH₃ | Y = NR⁹, R⁷, R⁹ = Et |
| V/VI.3 | CF₂H | H | CH₃ | Y = O, R⁷ = Et |
| V/VI.4 | CF₂H | H | CH₃ | Y = NR⁹, R⁷, R⁹ = Et |
| V/VI.5 | CF₂Cl | H | CH₃ | Y = O, R⁷ = Et |
| V/VI.6 | CF₂Cl | H | CH₃ | Y = NR⁹, R⁷, R⁹ = Et |
| V/VI.7 | CF₂Br | H | CH₃ | Y = O, R⁷ = Et |
| V/VI.8 | CF₂Br | H | CH₃ | Y = NR⁹, R⁷, R⁹ = Et |

The invention further concerns a process for the manufacture of a chemical composition according to the present invention, which comprises at least one of the steps of subjecting a chemical composition comprising a compound of formula (I) which has a content of compound of formula (I) of c2>c1, to at least one step selected from the group of steps consisting of:
i. crystallization
ii. washing
iii. chromatography
iv. salt formation, recovery of intermediary salt, and hydrolysis of salt
v. distillation.

In one aspect, the at least one step applied in the process for the manufacture of a chemical composition is a crystallization step, wherein the chemical composition comprising a compound of formula (I) which has a content of compound of formula (I) of c2>c1 is crystallized or re-crystallized from C₁-C₄-alcohols, preferably methanol or ethanol, or mixtures with water thereof. This is particularly advantageous when R³ is H. The at least one step applied in the process for the manufacture of a chemical composition can also be a washing step, wherein the chemical composition comprising a compound of formula (I) which has a content of compound of formula (I) of c2>c1 is washed with aromatic or aliphatic hydrocarbons, for example hexanes or petrolethers, or mixtures thereof. This is particularly advantageous when R³ is H. The at least one step applied in the process for the manufacture of a chemical composition can also be a chromatography step, wherein the chemical composition comprising a compound of formula (I) which has a content of compound of formula (I) of c2>c1 is chromatographed over a suitable carrier, e.g. silica, employing a suitable solvent, for example dichloromethane and/or THF, lower alcohols such as ethanol or methanol, aromatic or aliphatic hydrocarbons, for example hexane, heptane, cyclohexane, toluene, petrolether, and mixtures two or more of all foregoing solvents. This is particularly advantageous when R³ is selected from the group consisting of C₁-C₁₂-alkyl, C₂-C₆ alkenyl, aryl or C₃-C₈-cycloalkyl group, each of which is optionally substituted. In one aspect, the at least one step applied in the process for the manufacture of a chemical composition is a salt formation step with subsequent salt recovery and salt hydrolysis steps. In such a step sequence, a salt is formed from the compound of formula (I) contained in the chemical composition which has a content of compound of formula (I) of c2>c1, for example when R³ is H, by addition of a suitable base. The at least one step applied in the process for the manufacture of a chemical composition can also be a distillation step, wherein the chemical composition comprising a compound of formula (I) which has a content of compound of formula (I) of c2>c1 is distilled under suitable conditions, for example under reduced pressure and/or elevated temperatures This is particularly advantageous when R³ is selected from the group consisting of C₁-C₁₂-alkyl, C₂-C₆ alkenyl, aryl or C₃-C₈-cycloalkyl group, each of which is optionally substituted.

The invention also relates to a process for the manufacture of a chemical composition according to the present invention, which comprises at least one of the steps of subjecting a chemical composition comprising a compound of formula (I) which has a content of compound of formula (I) of c2>c1, to at least one of the steps selected from the group consisting of crystallization, washing, chromatography, salt formation, recovery of intermediary salt, and hydrolysis of salt and distillation, wherein the process further comprises at least one of the steps a) or b), wherein

a) is a step of reacting a compound of formula (XIII)

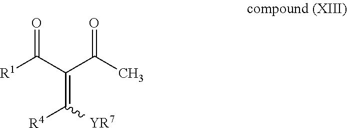

with a compound of formula (XIV), (XV) or (XVI)

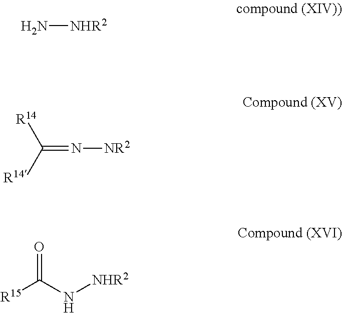

wherein Y is selected from the group consisting of S, O and NR⁹, wherein O and NR⁹ are preferred,

wherein R⁷ and R⁹ independently are selected from the group consisting of C₁-C₁₂-alkyl, C₂-C₆ alkenyl or C₃-C₁₀-cycloalkyl group, each of which is optionally substituted

or, when Y=NR⁹, R⁷ together with R⁹ and the nitrogen atom to which the two radicals are attached are an optionally substituted 5- to 10-membered heterocyclic radical which, in addition to the nitrogen atom, may contain a further 1, 2 or 3 heteroatoms selected from the group consisting of O, N and S as ring members,

wherein R² is defined as above,

wherein R¹⁴ and R^{14′} independently from each other in (XV) are selected from the group consisting of C₁-C₁₂-alkyl, C₂-C₆ alkenyl, cycloalkyl, aryl, heteroaryl, aralkyl, each of which is optionally substituted, R¹⁵ is selected from the group consisting of R¹⁶=C₁-C₁₂ alkyl, OR¹⁶ and NR¹⁷R^{17′}, wherein R¹⁷ and R^{17′} independently are selected from the group consisting of C₁-C₁₂-alkyl and H, and

step b) is a step of reacting the composition obtained by step a) with an aqueous solution comprising a hypohalite X″O⁻, wherein X″ is selected from Br, Cl and I, i.e. the halohalite is selected from the group consisting of BrO⁻, ClO⁻ or IO⁻. In one particular aspect of the present invention, when the process comprises step a) and step b), the at least one step selected from the group consisting of crystallization, washing, chromatography, salt formation, recovery of intermediary salt, and hydrolysis of salt and distillation is applied after step a) and before step b). In this case, this is particularly suitable when a step of i. crystallization is applied between a) and b), in particularly crystallization from petrolether. Depending on the reaction conditions of step b), compounds of formula (XIX) and/or (XX), wherein R¹ and R⁴ are defined as above, and X″ is selected from the group consisting of Br, Cl and I, are formed, in particular at elevated temperatures and/or acidic conditions

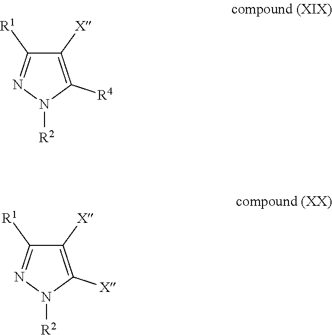

Compound (XIX) and/or compound (XX) can also be comprised in the chemical composition according to the present invention.

The invention further relates to a process for the manufacture of an agrochemical or pharmaceutical compound, which comprises the step of reaction of the chemical composition according to the present invention, wherein the chemical composition comprises equal to or more than 95 w % (content c1) of compound of formula (I), with at least one amine of formula (VI) NR¹²HQ, wherein R¹² is selected from the group consisting of H, C₁-C₁₂-alkyl, C₂-C₆ alkenyl or C₃-C₈-cycloalkyl group, wherein H and C₁-C₄-alkyl are preferred, and wherein Q is an optionally substituted aryl or heteroaryl group. The aryl or heteroaryl group can also be bi- or tricyclic, wherein one or more rings which are bound to the aryl or heteroaryl group can be non-aromatic. Generally, Q is selected from the group consisting of phenyl, naphtalene, 1,2,3,4-tetrahydronaphthalene, 2,3-dihydro-1H-indene, 1,3-dihydroisobenzofuran, 1,3-dihydrobenzo[c]thiophene, 6,7,8,9-tetrahydro-5H-benzo[7]annulene, thiophene, furan, thioazole, thiadiazole, oxazole, oxadiazole, pyridine, pyrimidine, triazine, tetrazine, thiazine, azepine and diazepine, each of which is optionally substituted. Particularly suitable groups Q are Q1,

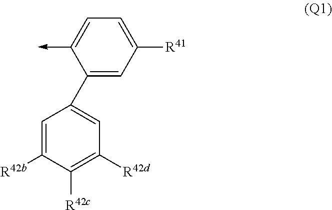

wherein R⁴¹, R^{42b}, R^{42c} and R^{42d} are each, independently, hydrogen or halogen, said halogen is especially chlorine or fluorine;

Q2, Q3 and Q4.

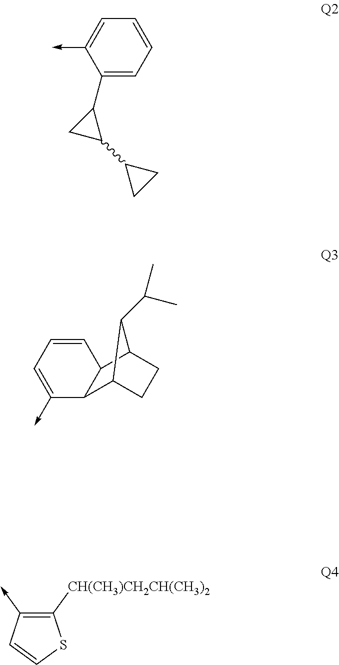

In one aspect, the process for the manufacture of an agrochemical or pharmaceutical compound, which comprises the step of reacting the chemical composition comprising equal to or more than 95 w % (content c1) of compound of formula (I), with at least one amine of formula (VI) NR¹²HQ, wherein R¹² is selected from the group consisting of C₁-C₁₂-alkyl, C₂-C₆ alkenyl, aryl or C₃-C₈-cycloalkyl group, each of which is optionally substituted, the step is performed in the presence of a base or a Lewis acid, preferably an aluminum or boron halide. The principles of such a step are disclosed, for example, in WO2012/055864 and WO2016/016298.

In another aspect, the process for the manufacture of an agrochemical or pharmaceutical compound, which comprises the step of reacting the chemical composition comprising equal to or more than 95 w % (content c1) of compound of formula (I), with at least one amine of formula (VI) NR¹²HQ, wherein R¹² is selected from the group consisting of H, C₁-C₁₂-alkyl, C₂-C₆ alkenyl, aryl or C₃-C₈-cycloalkyl group, each of which is optionally substituted, the process further comprises the step of converting the compound of formula (I) into a compound of formula (XII)

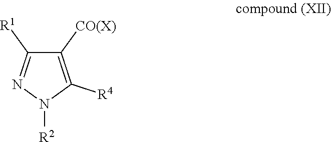

wherein X is selected from the group consisting of halogen, preferably F, Cl or Br, and —O—C(O)—R¹³, wherein R¹³ is selected from the group consisting of C₁-C₁₂-alkyl, C₂-C₆ alkenyl, cycloalkyl, aryl, heteroaryl, aralkyl, each of which is optionally substituted. Often, when R¹² is H, the conversion of the compound of formula (I) into the compound of formula (XII) is effected by treatment of (I) with thionyl chloride, phosphorous trichloride, phosphorous pentabromide or cyanuric fluoride. When R¹² is selected from the group consisting of C₁-C₁₂-alkyl, C₂-C₆ alkenyl, aryl or C₃-C₈-cycloalkyl group, each of which is optionally substituted, prior to conversion into (XII), compound (I) can be subjected to a step of acidic or basic hydrolysis to convert R¹² to H. As above, Q is selected from the group consisting of phenyl, naphtalene, 1,2,3,4-tetrahydronaphthalene, 2,3-dihydro-1H-indene, 1,3-dihydroisobenzofuran, 1,3-dihydrobenzo[c]thiophene, 6,7,8,9-tetrahydro-5H-benzo[7]annulene, thiophene, furan, thioazole, thiadiazole, oxazole, oxadiazole, pyridine, pyrimidine, triazine, tetrazine, thiazine, azepine and diazepine, each of which is optionally substituted. Particularly suitable groups Q are Q1, Q2, Q3 and Q4.

In another aspect, the process for the manufacture of an agrochemical or pharmaceutical compound, which comprises the step of reacting the chemical composition comprising equal to or more than 95 w % (content c1) of compound of formula (I), with at least one amine of formula (VI) NR¹²HQ, wherein R¹²=H, the reaction is performed in the presence of Dicyclohexylcarbodiimide (Steglich esterification) or an auxiliary compound selected from the group consisting of triphenylphosphane, DEAD (diethylazodicarboxylate) or DIAD (diisopropylazodicarboxylate) (Mitsunobu reaction).The process can further comprise a step wherein compound (I) is subjected to a step of acidic or basic hydrolysis to convert R¹² to H. The preferred compounds of Q are defined as above.

The invention further concerns the use of the chemical composition according to the present invention for the manufacture of an agrochemical or pharmaceutical compound.

The compounds of formula (I), and compositions comprising (I) and at least one of the compounds (II) to (IX), (XVIIIa), (XVIIIb), (XIX), (XX), (X) and optionally (XI) can be obtained by reaction of compounds of formula (XIII) with compounds of formula (XIV), (XV) or (XVI), wherein a compound of formula (XIV) with R²=Me is preferred. Compounds of formula (XIII) can be obtained by reaction of alkenone derivatives XVII with acyl halides or carboxylic anhydrides.

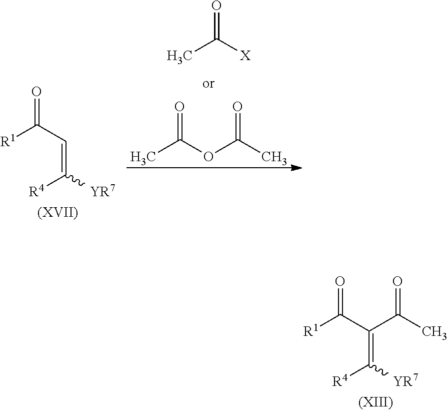

Compounds of formula (XVII) can be obtained by reaction of vinyl ethers with acyl halides, and optional reaction with an amine NHR⁹R⁷.

Similarly, (XIII) can be obtained by the following reaction:

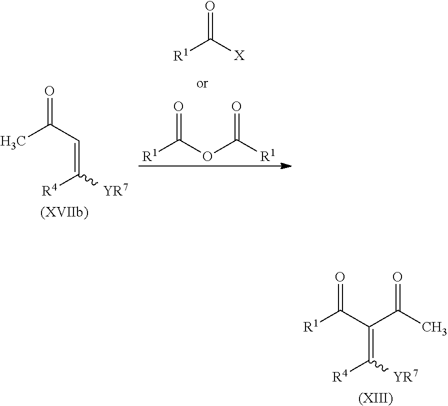

When compound (XVII) is used in the manufacture of the chemical composition, residual (XVII) can lead to compounds of formula (XVIIIa) and/or formula (XVIIIb), wherein R¹, R² and R⁴ are defined as above, when reacted with a compound of formula (XIV), (XV) or (XVI).

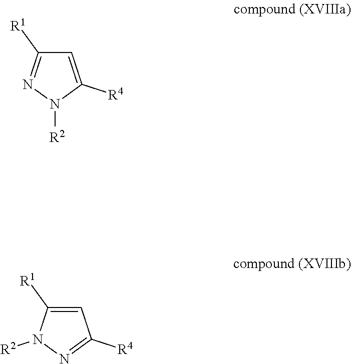

Compounds (XVIIIa) and/or (XVIIIb) can also be comprised in the chemical composition according to the present invention.

A composition comprising a compound of formula (I) and at least one of the compounds selected from (III), (IV), (V) and (VI) and optionally at least one of (XVIIIa) and (XVIIIb) can also be obtained by the reaction of a compound of formula (XXI) with a compound of formula (XIV), (XV) or (XVI). Reaction of a compound of formula (XXI) with a compound of formula (XIV), (XV) or (XVI) is then often followed by treatment with a base, such as aq. NaOH, KOH or Ca(OH)₂. R⁵ in (XXI) preferably is selected from the group consisting of CBr₃, CCl₃, CI₃ and CF₃, wherein CCl₃ and CF₃ are preferred.

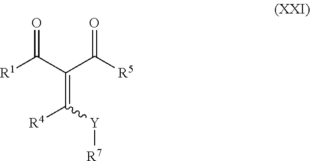

A compound of formula (XXI) can be obtained by a similar reaction as compound (XIII) from (XVII) or (XVIIb), when CH3 in (XVII) or (XVIIb) and the acid halide or acid anhydride added thereto is replaced with R⁵ respectively.

The present invention will be described in detail by the following examples, without, however, being limited thereto.

### EXAMPLES

### Example 1: Manufacture of 4-(dimethylamino)but-3-en-2-one (DMAA)

50 g DMFDMA (dimethylformamide dimethylacetal) was dissolved in 350 ml technical acetone and mixture heated in pressure reactor at 100° C. for 24 h. Resulted solution was evaporated and crude material (79% GC purity) distilled under vacuum (0.1 mbar)

Main fraction collected at 50-60° C. Yield 37 g, 78%, yellow crystals upon refrigeration.

### Example 2: Manufacture of 3-(dimethylamino)methylene-1,1-difluoro-2,4-pentadione

DMAA from example 1 (11.54 g, 0.1 mol) was dissolved in 100 ml of DCM and trimethylamine (14.2 ml, 0.1 mol) added. The mixture was cooled down to −30° C. and DFAF (difluoroacetylchloride, 10 g, 0.1 mol) was introduced as gas. The mixture was allowed to warm to 21° C. and stirred overnight. The mixture was extracted with 50 ml cold water and 50 ml brine, and the organic phase dried over Na2SO4. The volatiles were evaporated.

Yield 19.4 g red oil, 97.2%, crystallizes in the fridge.

### Example 3: Manufacture of 3-(difluoromethyl)-1-methyl-1H-pyrazol-4-carboxylic acid

40% aqueous MMH (monomethylhydrazine, 0.022 mol) was cooled down to −25° C. To this, 4.2 g (0.022 mol) the compound of example 2 in 10 ml dichloromethane was drop wise added maintaining −25° C. temperature. Mixture was stirred further at the same temperature for 30 min, then allowed to warm to RT. Mixture was washed with 20 ml brine, organic phase dried over Na2SO4 and evaporated. The solids were washed with 3×50 ml hot hexane (suspended in hot hexane, then cooled down, then filtered). 5 g of the solids were added portion wise to a solution of 2.3 g NaOH (2 eq) in 50 mL 15% NaOCl while maintained the temperature between 25-30° C. The mixture was stirred for approximately 6 hours until a clear yellow solution was obtained. The mixture was acidified with 1N HCl and cooled with ice. The resulting suspension was filtered and washed with cold water. 4.5 g of the desired product was obtained. The product comprised 0.1% of an impurity according to the invention as determined by ¹H-NMR.

1. A chemical composition comprising a compound of formula (I) 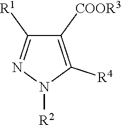
wherein
R¹ is a halogenated C₁₋₄ alkyl group, or R¹ is the group —C(O)H
R² is selected from the group consisting of H, C₁-C₁₂-alkyl, C₂-C₆ alkenyl, cycloalkyl, aryl, heteroaryl, and aralkyl, each of which is optionally substituted,
R³ is selected from the group consisting of H, C₁-C₁₂-alkyl, C₂-C₆ alkenyl, aryl and C₃-C₈-cycloalkyl group, each of which is optionally substituted and
R⁴ is selected from the group consisting of H, X′, COOR′, OR′, SR′, and C(O)NR′₂, wherein each R′ is independently hydrogen, CN, C₁-C₁₂-alkyl, C₂-C₆ alkenyl, aryl, cycloalkyl, aralkyl, or heteroaryl, each of which is optionally substituted;
and at least one compound selected from the group consisting of:
Formula (II) 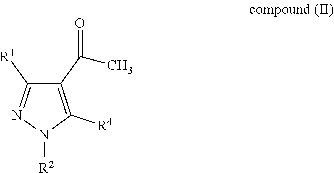
Formula (III) 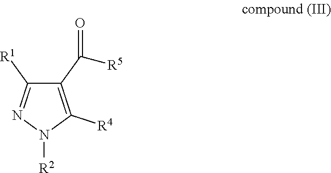
wherein R⁵ is a halogenated methyl group, or wherein R⁵ is the group N(R²)₂, wherein the two R² groups can be independently selected from the group defined above for R²
Formula (IV) 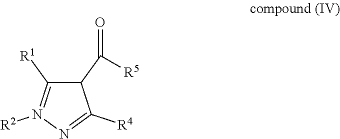
wherein R¹, R², R⁴ and R⁵ have the same meaning as above,
Formula (V) 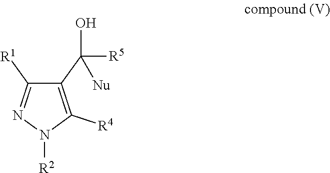
wherein R¹, R², R⁴ and R⁵ have the same meaning as above, and wherein Nu is a nucleophilic group,
Formula (VI) 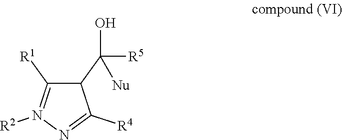
wherein R¹, R², R⁴, R⁵ and R⁶ have the same meaning as above,
Formula (VII) 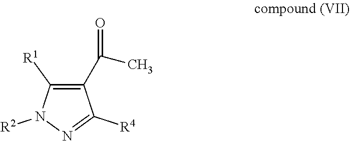
wherein R¹, R² and R⁴ have the same meaning as above,
Formula (VIII) 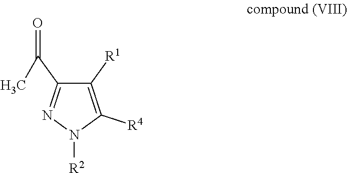
wherein R¹, R² and R⁴ have the same meaning as above,
Formula (IX) 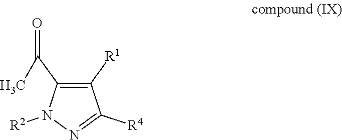
wherein R¹, R² and R⁴ have the same meaning as above,
Formula (XVIIIa) 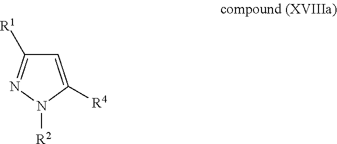
wherein R¹, R² and R⁴ are defined as above,
Formula (XVIIIb) 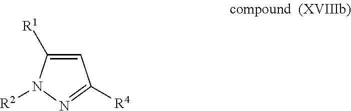
wherein R¹, R² and R⁴ are defined as above,
Formula (XIX) 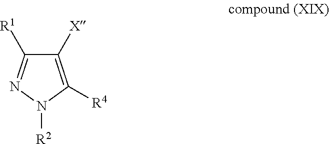
wherein R¹ and R⁴ are defined as above, and X″ is selected from the group consisting of Br, Cl and I,
Formula (XX) 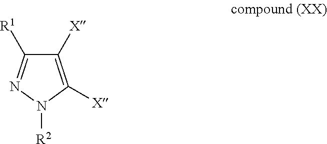
wherein R¹ is defined as above, and X″ is independently selected from the group consisting of Br, Cl and I,
Formula (X) 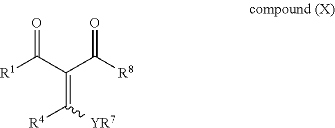
wherein R¹ and R⁴ are described as above, R⁸ is R⁵ or CH₃, Y is selected from the group consisting of S, O and NR⁹,
Wherein R⁷ and R⁹ independently are selected from the group consisting of C₁-C₁₂-alkyl, C₂-C₆ alkenyl and C₃-C₁₀-cycloalkyl group, each of which is optionally substituted,
or, when Y=NR⁹, R⁷ together with R⁹ and the nitrogen atom to which the two radicals are attached are an optionally substituted 5- to 10-membered heterocyclic radical which, in addition to the nitrogen atom, may contain a further 1, 2 or 3 heteroatoms selected from the group consisting of O, N and S as ring members;
and optionally a compound of formula (XI) 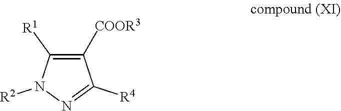
wherein R¹, R² and R⁴ have the same meaning as above, wherein the content of (XI), if present, is equal to or less than 0.9%,
wherein the chemical composition comprises equal to or more than 95 w % (content c1) of compound of formula (I).

2. The chemical composition according to claim 1, wherein the chemical composition comprises equal to or more than 98 w % (content c1) of compound of formula (I).

3. The chemical composition according to claim 1, wherein the chemical composition comprises equal to or more than 99 w % (content c1) of compound of formula (I).

4. The chemical composition according to claim 1, wherein the cumulative amount of compounds (II) to (XI) comprised in the chemical composition is equal to or less than 5 w %.

5. The chemical composition according to claim 1, wherein the cumulative amount of compounds (II) to (XI) comprised in the chemical composition is equal to or less than 10,000 ppmw.

6. The chemical composition according to claim 1, wherein the individual amount of compounds (II) to (XI) comprised in the chemical composition is equal to or less than 1,000 ppmw.

7. A process for the manufacture of a chemical composition according to claim 1, which comprises subjecting a chemical composition comprising a compound of formula (I) which has a content of compound of formula (I) of c2>c1, to at least one step selected from the group of steps consisting of:
i. crystallization,
ii. washing,
iii. chromatography,
iv. salt formation, recovery of intermediary salt, and hydrolysis of salt, and
v. distillation.

8. The process according to claim 7, wherein the process further comprises at least one of the steps a) or b), wherein
step a) is a step of reacting a compound of formula (XIII) 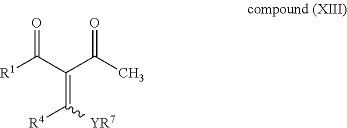
with a compound of formula (XIV), (XV) or (XVI) 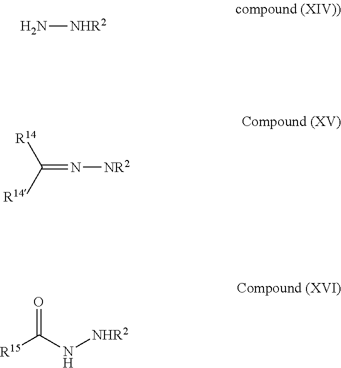
wherein Y is selected from the group consisting of S, O and NR⁹,
wherein R⁷ and R⁹ independently are selected from the group consisting of C₁-C₁₂-alkyl, C₂-C₆ alkenyl and C₃-C₁₀-cycloalkyl group, each of which is optionally substituted,
or, when Y=NR⁹, R⁷ together with R⁹ and the nitrogen atom to which the two radicals are attached are an optionally substituted 5- to 10-membered heterocyclic radical which, in addition to the nitrogen atom, may contain a further 1, 2 or 3 heteroatoms selected from the group consisting of O, N and S as ring members,
wherein R² is defined as above,
wherein R¹⁴ and R^{14′} independently from each other in (XV) are selected from the group consisting of C₁-C₁₂-alkyl, C₂-C₆ alkenyl, cycloalkyl, aryl, heteroaryl, and aralkyl, each of which is optionally substituted,
R¹⁵ is selected from the group consisting of R¹⁶, OR¹⁶ and NR¹⁷R^{17′}, wherein R¹⁶ is C₁-C₁₂-alkyl, and R¹⁷ and R^{17′} independently are selected from the group consisting of C₁-C₁₂-alkyl and H,
step b) is a step of reacting the composition obtained by step a) with an aqueous solution comprising a hypohalite, wherein the hypohalite is BrO⁻, ClO⁻ or IO⁻.

9. A compound according to any one of formula (III) to (IX),
Formula (III) 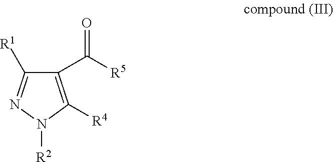
wherein
R¹ is a halogenated C₁₋₄ alkyl group, or R¹ is the group —C(O)H
R² is selected from the group consisting of H, C₁-C₁₂-alkyl, C₂-C₆ alkenyl, cycloalkyl, aryl, heteroaryl, and aralkyl, each of which is optionally substituted,
R³ is selected from the group consisting of H, C₁-C₁₂-alkyl, C₂-C₆ alkenyl, aryl and C₃-C₈-cycloalkyl group, each of which is optionally substituted and
R⁴ is selected from the group consisting of H, X′, COOR′, OR′, SR′, and C(O)NR′₂, wherein each R′ is independently hydrogen, CN, C₁-C₁₂-alkyl, C₂-C₆ alkenyl, aryl, cycloalkyl, aralkyl, or heteroaryl, each of which is optionally substituted,
wherein R⁵ is a halogenated methyl group, or wherein R⁵ is the group N(R²)₂, wherein the two R² groups can be independently selected from the group defined above for R²
Formula (IV) 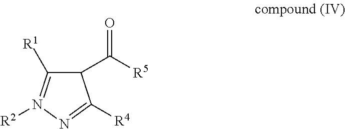
wherein R¹, R², R⁴ and R⁵ have the same meaning as above,
Formula (V) 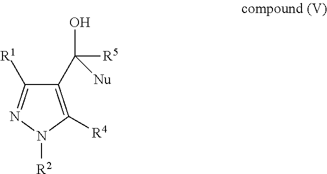
wherein R¹, R², R⁴ and R⁵ have the same meaning as above, and wherein Nu is a nucleophilic group,
Formula (VI) 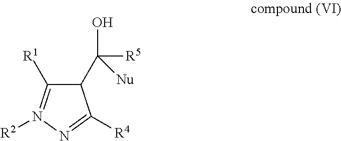
wherein R¹, R², R⁴, R⁵ and R⁶ have the same meaning as above,
Formula (VII) 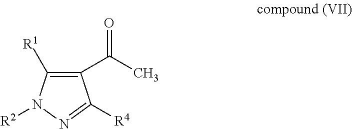
wherein R¹, R² and R⁴ have the same meaning as above,
Formula (VIII) 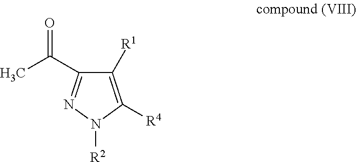
wherein R¹, R² and R⁴ have the same meaning as above,
Formula (IX) 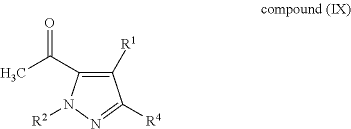
wherein R¹, R² and R⁴ have the same meaning as above,
with the proviso that in compound (III) and (X) R⁵ is a monohalo- or dihalomethyl group or wherein R⁵ is the group N(R²)₂, wherein the two R² groups can be independently selected from the group defined above for R².

10. A process for the manufacture of an agrochemical or pharmaceutical compound, which comprises the step of reacting the chemical composition according to claim 1 with at least one amine of formula NR¹²HQ, wherein R¹² is selected from the group consisting of H, C₁-C₁₂-alkyl, C₂-C₆ alkenyl and C₃-C₈-cycloalkyl group, and wherein Q is an optionally substituted aryl or heteroaryl group.

11. The process according to claim 10, which comprises subjecting the chemical composition comprising a compound of formula (I) which has a content of compound of formula (I) of c2>c1, to at least one step selected from the group of steps consisting of:
i. crystallization,
ii. washing,
iii. chromatography,
iv. salt formation, recovery of intermediary salt, and hydrolysis of salt, and
v. distillation.

12. The process according to claim 10, wherein R³ is selected from the group consisting of C₁-C₁₂-alkyl, C₂-C₆ alkenyl, aryl and C₃-C₈-cycloalkyl group, each of which is optionally substituted, and wherein the step is performed in the presence of a base or a Lewis acid.

13. The process according to claim 10, wherein R³ is selected from the group consisting of H, C₁-C₁₂-alkyl, C₂-C₆ alkenyl, aryl and C₃-C₈-cycloalkyl group, each of which is optionally substituted,
and wherein the process comprises the step of converting the compound of formula (I) into a compound of formula (XII) 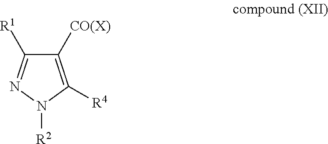
wherein X is selected from the group consisting of halogen, and —O—C(O)—R¹³, wherein R¹³ is selected from the group consisting of C₁-C₁₂-alkyl, C₂-C₆ alkenyl, cycloalkyl, aryl, heteroaryl, and aralkyl, each of which is optionally substituted;
or wherein R³=H and the reaction is performed in the presence of Dicyclohexylcarbodiimide or an auxiliary compound selected from the group consisting of triphenylphosphane, DEAD (diethylazodicarboxylate) and DIAD (diisopropylazodicarboxylate).

14. The process according to claim 10, wherein the process comprises the step of converting the compound of formula (I), wherein R³ is selected from the group consisting of C₁-C₁₂-alkyl, C₂-C₆ alkenyl, aryl and C₃-C₈-cycloalkyl group, each of which is optionally substituted, to a compound of formula (I), wherein R³=H, wherein the step is an acidic or basic hydrolysis.

15. (canceled)

16. The chemical composition according to claim 1, wherein Y is selected from the group consisting of O and NR⁹.

17. The process according to claim 8, wherein Y is selected from the group consisting of O and NR⁹.

18. The process according to claim 10, wherein R¹² is selected from the group consisting of H and C₁-C₄-alkyl.

19. The process according to claim 12, wherein the Lewis acid is an aluminum or boron halide.

20. The process according to claim 13, wherein the halogen is F, Cl or Br.
